(19) United States
(12) Patent Application Publication
(10) Pub. No.: US 2023/0095832 A1
(43) Pub. Date: Mar. 30, 2023

(54) ADHESION BARRIER COMPOSITION, METHODS OF FABRICATION AND USE THEREOF

(71) Applicant: SPOROGENICS PTE. LTD., Singapore (SG)

(72) Inventor: WeiBeng NG, Singapore (SG)

(21) Appl. No.: 17/908,841

(22) PCT Filed: Mar. 1, 2021
(86) PCT No.: PCT/SG2021/050099

§ 371 Date: Sep. 1, 2022

(30) Foreign Application Priority Data
Mar. 2, 2020 (SG) 10202001864Y

(51) Int. Cl. A61K 36/18; A61K 47/54; A61K 36/82; A61K 31/225; A61K 31/728; A61K 31/734; A61K 9/06; A61L 31/04

(52) U.S. Cl. CPC A61K 36/18; A61K 47/543; A61K 36/82; A61K 31/225; A61K 31/728; A61K 31/734; A61K 9/06; A61L 31/042

(57) ABSTRACT

The present invention relates, in general terms, to methods of reducing the formation of post-surgery tissue adhesion in a subject in need thereof by administering an adhesion barrier composition. The present invention also relates to methods of preventing the formation of tissue adhesion during or post-surgery in a subject in need thereof. The adhesion barrier composition comprises sporopollenin and a biodegradable polymer. The present invention also relates to methods of fabricating the adhesion barrier composition.

### TECHNICAL FIELD

The present invention relates, in general terms, to methods of reducing or preventing the formation of surgical adhesion through the administration of an adhesion barrier composition. The present invention also relates to the adhesion barrier composition and methods of fabrication thereof.

### BACKGROUND

A surgical adhesion is a band of scar tissue that joins two internal body surfaces that are not usually connected, and may appear as thin sheets of tissue similar to plastic wrap, or as thick fibrous bands. This is a result of repair cells within the body not being able to tell the difference between one organ and another. In this regard, adhesions can develop as the body attempts to repair itself after surgery, infection, injury (trauma) or radiation. Adhesions typically form when two or more surfaces, such as the surfaces of discrete tissues, stick together at the site of surgery during the natural healing process. Adhesions can occur following virtually all types of surgeries and even the most careful and skilled surgeons find it difficult to avoid the formation of adhesions. In abdominal and pelvic surgery, adhesion formation is regarded as the most common postsurgical complication, occurring in 90% of procedures. Abdominal adhesions also occur in 10% of people who have never had surgery.

Up to 90 percent of women suffer post-operative adhesions following major gynaecological surgery. Adhesions can also affect the female reproductive organs (ovaries, fallopian tubes), the bowel, the area around the heart, the spine and the hand. They can cause a range of problems including infertility, dyspareunia (painful intercourse), pelvic pain and bowel obstruction or blockage.

Adhesions can delay recovering following a surgery and cause complications in subsequent surgeries. For example, leaks, wound infections and haemorrhages are common in people with adhesion-related perforations. Adhesion can also lead to other risks and complications, including small bowel obstructions, infertility and chronic pelvic pain.

Adhesions can also lead to a complex set of problems called adhesion-related disorder (ARD). ARD is a group of symptoms that may occur as a result of adhesions. A person with ARD will usually experience chronic abdominal pain.

The prevention of adhesions is an important goal of surgical practice. Many approaches to prevent adhesions have been suggested, but they either have not generally withstood rigorous clinical examination or they have major practical limitations. One such approach is to perform an operation to remove the adhesions. However, many times these operations are not effective because the adhesions simply reform.

Traditionally, drugs and materials such as animal membranes, gold foil, mineral oil, sheets made of rubber and Teflon have been used to reduce the risk of adhesion formation. However, reports of these results in reducing adhesions have not been encouraging and in this regard, it is accepted in the art that adhesions do occur and appear to be, to some degree, an almost unavoidable consequence of surgery, in particular abdominal and pelvic surgery.

Current adhesion barriers are not ideal. For example, Seprafilm (made by Sanofi) is a adhesion barrier indicated for use in patients undergoing abdominal or pelvic laparotomy as an adjunct intended to reduce the incidence, extent and severity of postoperative adhesions between the abdominal wall and the underlying viscera such as omentum, small bowel, bladder and stomach, and between the uterus and surrounding structures such as tubes and ovaries, large bowel and bladder. However, the incidence of adhesion is still reported to be high at 49% with the use of Seprafilm. Another product, Interceed (made by Johnson & Johnson) is a knitted fabric composed of a modified cellulose that swells and eventually gels after being placed on the injured site. However, this product requires a bloodless field and can migrate away from its intended position.

It would be desirable to overcome or ameliorate at least one of the above-described problems, or at least to provide a useful alternative.

### SUMMARY

The present invention relates to a method of reducing the formation of post-surgery tissue adhesion in a subject in need thereof by administering an adhesion barrier composition, the adhesion barrier composition comprising:

a) sporopollenin; and

b) a biodegradable polymer;

wherein the weight ratio of sporopollenin is about 1 wt/wt % to about 80 wt/wt % relative to the total composition.

The present invention also relates to a method of preventing the formation of tissue adhesion during or post-surgery in a subject in need thereof by administering an adhesion barrier composition, the adhesion barrier composition comprising:

a) sporopollenin; and

b) a biodegradable polymer;

wherein the weight ratio of sporopollenin is about 1 wt/wt % to about 80 wt/wt % relative to the total composition.

Sporopollenin is degradable in the physiological medium after a few weeks. In this regard, when in combination with the biodegradable polymer, the adhesion barrier composition is totally biodegradable and a secondary surgery to remove the composition is unnecessary. The adhesion barrier composition keeps tissue surfaces separated during the early days of wound healing after surgery and accordingly can reduce or prevent the formation of adhesion.

In some embodiments, the surgery is selected from peritoneal, pericardial, obstetric, gynaecological, neurosurgical, arthroscopic, orthopaedic, plastic, reconstructive, muscle or tendon surgery.

The present invention also relates to an adhesion barrier composition for use in reducing the formation of post-surgery tissue adhesion in a subject in need thereof, the adhesion barrier composition comprising:

a) sporopollenin; and

b) a biodegradable polymer;

wherein the weight ratio of sporopollenin is about 1 wt/wt % to about 80 wt/wt % relative to the total composition.

The present invention also relates to an adhesion barrier composition for use in preventing the formation of tissue adhesion during or post-surgery in a subject in need thereof, the adhesion barrier composition comprising:

a) sporopollenin; and

b) a biodegradable polymer;

wherein the weight ratio of sporopollenin is about 1 wt/wt % to about 80 wt/wt % relative to the total composition.

The present invention also relates to use of an adhesion barrier composition in the manufacture of a medicament for reducing the formation of post-surgery tissue adhesion in a subject in need thereof, the adhesion barrier composition comprising:

a) sporopollenin; and

b) a biodegradable polymer;

wherein the weight ratio of sporopollenin is about 1 wt/wt % to about 80 wt/wt % relative to the total composition.

The present invention also relates to use of an adhesion barrier composition in the manufacture of a medicament for preventing the formation of tissue adhesion during or post-surgery in a subject in need thereof, the adhesion barrier composition comprising:

a) sporopollenin; and

b) a biodegradable polymer;

wherein the weight ratio of sporopollenin is about 1 wt/wt % to about 80 wt/wt % relative to the total composition.

The present invention relates to an adhesion barrier composition comprising:

a) sporopollenin; and

b) a biodegradable polymer;

wherein the weight ratio of sporopollenin is about 1 wt/wt % to about 80 wt/wt % relative to the total composition.

In some embodiments, the weight ratio of sporopollenin is about 20 wt/wt % to about 80 wt/wt % relative to the total composition.

In some embodiments, sporopollenin comprises a polymer having ester bonds, wherein the sporopollenin is characterised with having at least 2% of the ester bond hydrolysed.

In some embodiments, sporopollenin comprises a polymer having ether bonds, wherein the sporopollenin is characterised with having at least 2% of the ether bond hydrolysed.

In some embodiments, sporopollenin comprises a polymer having peptide bonds, wherein at least 2% of the peptide bond (amide bond) is hydrolysed.

In some embodiments, sporopollenin comprises a lipid, wherein at least 2% of the lipid is saponificated.

In some embodiments, the sporopollenin is derived from a pollen, the pollen originating from a flowering plant of the genus Baccharis, Helianthus or Camellia, Coreopsis, Callistephus, Dahalia, Solidago or a combination thereof.

In some embodiments, the sporopollenin has a particle size of about 20 μm to about 50 μm.

In some embodiments, the biodegradable polymer is selected from poly(glycolic) acid, polylactic acid, polydioxanone, polycaprolactone, calcium alginate, sodium hyalurate, hyaluronic acid or a combination thereof.

In some embodiments, the adhesion barrier composition further comprises a solvent.

The solvent can improve the dispersion of sporopollenin in the composition and can aid its application on a surface.

In some embodiments, the solvent is an aqueous medium.

In some embodiments, the adhesion barrier composition further comprises a lipid.

The lipid can improve the dispersion of sporopollenin in the composition. The lipid also increases the adhesion of composition to the surface.

In some embodiments, the lipid is selected from lauric acid, palmitic acid, α-linolenic acid, myristic acid, stearic acid, oleic acid, linoleic acid, eicosenoic acid, glycerides or a combination thereof.

In some embodiments, the adhesion barrier composition further comprises pollenkitt.

In some embodiments, the pollenkitt is selected from an angiosperm.

In some embodiments, the adhesion barrier composition further comprises intine.

In some embodiments, the intine is selected from polysaccharides, cellulose, hemicellulose and pectin, or a combination thereof.

In some embodiments, the adhesion barrier composition further comprises phenolic compound.

The phenolic compound can attenuate post-operative adhesion. The degree of inflammation and fibrosis can also be reduced.

In some embodiments, the phenolic compound is selected from gallic acid, caffeic acid, ferulic acid, p-coumaric, benzoic acid or a combination thereof.

In some embodiments, the weight ratio of the phenolic compound is at least 1 wt/wt % relative to the total composition.

In some embodiments, the adhesion barrier composition is capable of being completely resorbed after 6 months on application.

In some embodiments, the adhesion barrier composition is a film, gel or a liquid.

In some embodiments, when the adhesion barrier composition is an adhesion barrier film, the adhesion barrier film comprising:

a) a coating of sporopollenin; and

b) a biodegradable polymer film having two surfaces;

wherein the weight ratio of sporopollenin is about 1 wt/wt % to about 80 wt/wt % relative to the total composition; and

wherein the sporopollenin coats at least one surface of the biodegradable polymer film.

In some embodiments, the sporopollenin coats both surfaces of the biodegradable polymer film.

In some embodiments, the surface of the biodegradable polymer film is 100% covered by sporopollenin.

In some embodiments, the coating of sporopollenin has a thickness of about 50 μm to about 200 μm.

The present invention also relates to a method of fabricating an adhesion barrier composition, comprising:

a) dispersing a sporopollenin in or on a biodegradable polymer;

wherein the weight ratio of sporopollenin is about 1 wt/wt % to about 80 wt/wt % relative to the total composition.

### BRIEF DESCRIPTION OF THE DRAWINGS

Embodiments of the present invention will now be described, by way of non-limiting example, with reference to the drawings in which:

FIG. 1 illustrates (a) a pollen grain; and (b) the components of a pollen wall, sporoderm;

FIG. 2 illustrates an exemplary multilayered adhesion barrier film comprising (a) sporopollenin in the form of a continuous film alternating and/or sandwiched by/with continuous biodegradable polymer films;

FIG. 3 illustrates an exemplary method for forming the adhesion barrier composition;

FIG. 4 illustrates a surface morphology of sporopollenin;

FIG. 5A shows anti-adhesive scores with regard to the extent of adhesion for comparators relative to control; and

FIG. 5B shows anti-adhesive scores with regard to the severity of adhesion for comparators relative to control.

### DETAILED DESCRIPTION

Current technology for preventing adhesion is not ideal. For example, some adhesion barrier products require that surfaces need to be relatively bloodless or dry, otherwise the product will migrate or shift in position. Some products are difficult to handle laparoscopically. Other products need to be sutured in place. For example, Seprafilm is a hyaluronic acid carboxymethyl cellulose membrane and INTERCEED is an oxidized regenerated cellulose, both of which are frequently used in gynaecological surgery. However, problems remain with the use of these anti-adhesive agents. For example, studies have shown that in humans, Seprafilm reduces the severity of adhesions but it does not reduce incidence and has no effect on the prevention of pelvic adhesions in women. INTERCEED has been reported to have limitations in that blood infiltration renders the product completely ineffective for preventing adhesions. INTERCEED may increase the risk of adhesions if optimal haemostasis is not achieved. INTERCEED also provokes a significant leucocyte response and an inflammatory response may enhance adhesion.

In this regard, the present invention is predicated on the understanding that an ideal adhesion barrier needs to be efficient, safe, easy to use and cost effective. For example, in addition to the primary function of adhesion barrier to prevent or at least reduce direct contact to adjacent organs, an adhesion barrier should be made out of material that remains in the same place, does not disturb peritoneum regeneration (for example), allows haemostasis (because abundant fibrin formation accelerates adhesion at a bleeding site) and does not cause inflammation. It can also be beneficial for the adhesion barrier to have some initial adhesion to a surface so that it can be placed at a desired site easily without the use of sutures. It is postulated that an adhesion barrier which costs $150 and can reduce the adhesion related readmissions about 25% in one year can save more than $40 million over a 10-year period. This can be a substantial relief of the burden on a health care system.

The present invention relates to a method of reducing the formation of post-surgery tissue adhesion in a subject in need thereof by administering an adhesion barrier composition, the adhesion barrier composition comprising:

a) sporopollenin; and

b) a biodegradable polymer;

wherein the weight ratio of sporopollenin is about 1 wt/wt % to about 80 wt/wt % relative to the total composition.

In this regard, the method is capable of bringing the formation of post-surgery tissue adhesion to a more desirable state or condition, to a degree such that adhesion formation after administering the adhesion barrier composition is less than without the adhesion barrier composition.

In some embodiments, the reduction in adhesion formation is about 5%, about 10%, about 15%, about 20%, about 25%, about 30%, about 35%, about 40%, about 45%, about 50%, about 55%, about 60%, about 65%, about 70%, about 75%, about 80%, about 85%, about 90% or about 95%. In other embodiments, the reduction is about 5% to about 95%, about 10% to about 90%, about 20% to about 90%, about 30% to about 90%, about 40% to about 90%, about 50% to about 90%, about 60% to about 90%, or about 70% to about 90%.

The present invention also relates to a method of preventing the formation of tissue adhesion during or post-surgery in a subject in need thereof by administering an adhesion barrier composition, the adhesion barrier composition comprising:

a) sporopollenin; and

b) a biodegradable polymer;

wherein the weight ratio of sporopollenin is about 1 wt/wt % to about 80 wt/wt % relative to the total composition.

In this regard, the formation of adhesion is kept/stopped from happening. In other embodiments, adhesion formation is eliminated.

In some embodiments, the method is for reducing or preventing adhesion between abutting surfaces. The abutting surfaces can be selected from the group consisting of two or more surface portions of the same tissue and/or surfaces from each of two or more discrete tissues, and/or the surfaces of a tissue and implanted material. The meaning of the word “tissue” as used in this application is construed broadly to cover any internal part or organ of an organism.

The method can be used for any suitable surgery in which adhesions pose a risk. In some embodiments, the surgery is selected from peritoneal, pericardial, obstetric, gynaecological, neurosurgical, arthroscopic, orthopaedic, plastic, reconstructive, muscle or tendon surgery.

In other embodiments, the method also reduces inflammation or tissue damage in a subject in need thereof after post-surgery.

Embodiments of the methods as disclosed herein comprise a step of administering the adhesion barrier composition. For example, the adhesion barrier composition can be positioned between abutting surfaces in the course of surgery. The adhesion barrier composition can be positioned into place between abutting surfaces by any suitable means. Usually, the adhesion barrier composition is placed in a manner such that it separates the tissues of the body that have been traumatized by surgery from remaining healthier tissues of the body. Alternatively, the adhesion barrier composition is placed in a manner such that it separates two traumatized tissues of the body. In these and other embodiments, separation in this manner reduces the formation of adhesions between tissue surfaces.

In other embodiments, the adhesion barrier composition is positioned such that it extends beyond the incision or traumatized tissue. This facilitates its coverage around the tissue contours such that slippage is reduced or eliminated.

It was found that the adhesion barrier composition conforms well to moist tissues and can be used in the presence of blood. Alternatively, to further improve the adhesion of the adhesion barrier composition to the tissue, it may be sutured and/or stapled into place, or retained using any other suitable materials (such as adhesives) or methods. The surgical site can then be closed according to the standard surgical technique. It is envisioned that the adhesion barrier composition requires no modification of a surgical technique and does not negatively affect wound healing.

The present invention also relates to an adhesion barrier composition for use in reducing the formation of post-surgery tissue adhesion in a subject in need thereof, the adhesion barrier composition comprising:

a) sporopollenin; and

b) a biodegradable polymer;

wherein the weight ratio of sporopollenin is about 1 wt/wt % to about 80 wt/wt % relative to the total composition.

The present invention also relates to an adhesion barrier composition for use in preventing the formation of tissue adhesion during or post-surgery in a subject in need thereof, the adhesion barrier composition comprising:

a) sporopollenin; and

b) a biodegradable polymer;

wherein the weight ratio of sporopollenin is about 1 wt/wt % to about 80 wt/wt % relative to the total composition.

The present invention also relates to use of an adhesion barrier composition in the manufacture of a medicament for reducing the formation of post-surgery tissue adhesion in a subject in need thereof, the adhesion barrier composition comprising:

a) sporopollenin; and

b) a biodegradable polymer;

wherein the weight ratio of sporopollenin is about 1 wt/wt % to about 80 wt/wt % relative to the total composition.

The present invention also relates to use of an adhesion barrier composition in the manufacture of a medicament for preventing the formation of tissue adhesion during or post-surgery in a subject in need thereof, the adhesion barrier composition comprising:

a) sporopollenin; and

b) a biodegradable polymer;

wherein the weight ratio of sporopollenin is about 1 wt/wt % to about 80 wt/wt % relative to the total composition.

In another aspect, the present invention relates to an adhesion barrier composition comprising:

a) sporopollenin; and

b) a biodegradable polymer;

wherein the weight ratio of sporopollenin is about 1 wt/wt % to about 80 wt/wt % relative to the total composition.

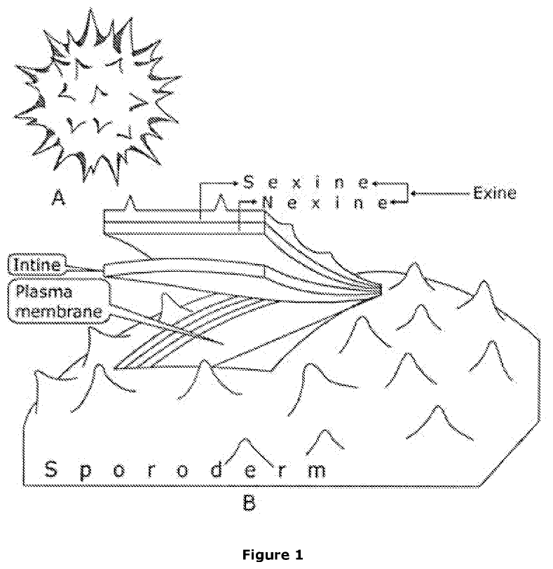

Pollen is the carrier of the male gametes or their progenitor cell of a plant. The pollen has a heterogeneously developed wall, and pollens can be distinguished by a multitude of sculptures and structures (FIG. 1). The pollen wall, the sporoderm, is the most complex wall system in the life cycle of higher plants. It includes three main domains that are highly different in their chemical composition, morphological structure, and physiological and biological significance. These domains are intine, exine, and pollen coat. The exine (the outer, resistant, and rigid layer) is richly sculptured and ornamented. It is composed mainly of sporopollenin, an organic biopolymer of extremely high stability. The outer pollen wall with its structures and surface substances takes part in the process of adaptation between the pollen and environment.

The intine layer is located on the inside of the exine and adjacent to the pollen plasma membrane. The intine comprises cellulose, hemicellulose, and pectin. The intine layer consists of two separate layers, the granular exintine with pectine and protein inclusions facing the exterior, and a microfibrillar cellulosic endintine facing the interior. The pectin network is known to interact with the cellulose network through both covalent ester linkages and non-covalent hydrogen bonding.

‘Sporopollenin’ refers to the major component of exine of a pollen grain. It is believed that sporopollenin is a polymer of a uniform composition, i.e. relatively small monomer moieties of similar primary structure are present. For example, the main structure of sporopollenin can be a simple aliphatic polymer containing aromatic conjugated side chains. Sporopollenin can also contain long chain fatty acids and xanthophylls. Sporopollenin is commonly arranged in complex forms in walls of pollen in such a way that it forms an elaborately sculptured characteristic pattern on sexine. This pattern can, for example, include spikes. To obtain sporopollenin, the pollen can be processed such that other components of the pollen are extracted, leaving an exine comprising mainly of sporopollenin. The exine can be further processed to leave behind a shell comprising mainly sporopollenin. As used herein, ‘sporopollenin’ refers to a component of the exine of a pollen. In this regard, sporopollenin, when as part of a pollen, can also be used in the adhesive barrier composition. Sporopollenin, when as part of sporoderm (intine, exine and pollen coat) can also be used in the adhesive barrier composition. Sporopollenin, when as part of intine and exine can also be used in the adhesive barrier composition. Alternatively, the sporopollenin can be extracted and isolated from pollen to form a microcapsule (exine shell or processed exine shell) and used in the adhesive barrier composition.

The inventor has found that it is beneficial for an adhesion barrier composition to have one or more of the following attributes: i) infiltration or attachment of cells or blood into the adhesion barrier composition should be avoided, ii) the adhesion barrier composition should be able to be attached at the desired site for a specified period of time, iii) a foreign body reaction should be minimized to reduce inflammation, iv) the biodegradation period should be able to be controlled, so that the barrier capacity can be sustained for a requisite period of time, v) the adhesion barrier composition should be flexible and have superior mechanical properties, including tensile strength and wet strength, for ease of handling during surgery, and vi) there should be no deformation for a necessary period of time, because the wound should be covered exactly.

In this regard, the inventor has found that a composition comprising sporopollenin can be used for forming an adhesion barrier composition. It is believed that sporopollenin contains two types of cross-linkages (ester and acetal/ether). These act as chemical clips that provide a pollen wall, sporoderm, with chemical and mechanical stability. Ester bonds are resistant to mildly acidic conditions, which prevents/slows down the pollen's degradation in the natural environment. However, in physiological medium, sporopollenin was found to be able to degrade after a few weeks. In this regard, when in combination with the biodegradable polymer, a secondary surgery to remove the composition is unnecessary and hence provides greater ease of use, and decreased inconvenience and risk of surgical complications for a patient.

In at least some embodiments, the sporopollenin used in the adhesion barrier composition retains its sculptured structure. For example, the sculptured surface of sporopollenin increases a surface area for contact with a biological surface. This is beneficial for improving the adhesion of the biological surface to the adhesion barrier composition, such that adhesion (between biological surfaces) does not occur. In this regard, the use of sporopollenin in the adhesion barrier composition can increase the efficiency of preventing adhesion.

The incorporation of the sporopollenin into a biodegradable polymer at a suitable concentration allows the composition to be flexible (at least when being applied to the targeted region) and have acceptable mechanical properties, including tensile strength and wet strength, for ease of handling during surgery. This also reduces the tendency of the applied composition to deform for a necessary period of time.

In some embodiments, the sporopollenin is derived from a pollen. The pollen can originate from a flowering plant of the genus Baccharis, Helianthus or Camellia, Coreopsis, Callistephus, Dahalia, Solidago, or a combination thereof.

The sporopollenin can be an extract from a pollen. In this regard, the extract can also contain other components of the pollen. For example, the sporopollenin can be from a pollen extract which also contains intine or a part thereof.

In some embodiments, the sporopollenin is a part of a pollen. The pollen can originate from a flowering plant of the genus Baccharis, Helianthus or Camellia, Coreopsis, Callistephus, Dahalia, Solidago or a combination thereof.

It was found that sporopollenin from the genus of Helianthus may be used. For example, the species Helianthus annuus can be used. The pollen from Helianthus has a grain size of about 40 μm to about 50 μm. When the pollen is processed and sporopollenin of Helianthus annuus obtained, sporopollenin has a natural morphology of spikes on its surface and accordingly provides for a ‘sticky’ attribute. There is thus no need to perform suture of the adhesion barrier composition onto the site during operation, and also can reduce the potential of migration off-site. Further, the spikes inherently present on this sporopollenin enables good cross-linking between and within the sporopollenin and the biodegradable polymer such that a uniform, continuous material can be formed. Accordingly, an adhesive is not required.

It was also found that when the adhesion barrier composition comprising sporopollenin is placed on a biological surface or tissue, the tissue can provide a platform for entangling the sporopollenin. In this regard, collagen fibrils may grow from the tissue to improve the adhesion of the tissue to sporopollenin, and to the adhesion barrier composition.

As sporopollenin is a biomaterial, it is believed to be safe and non-toxic. Further, due to the chemical moieties on the surface of sporopollenin, it is believed that inflammation and fibrosis can also be reduced, thus leading to a ‘cleaner’ healing.

The pollen may be defatted before sporopollenin is used in the adhesion barrier composition. It is generally beneficial to use pollen that has been defatted before use.

The defatting step may be carried out by dispersing the pollen in an organic solvent, such as, without limitation, acetone or other ethers or ketones, such as diethyl ether, methyl ethyl ketone and the like. In principle, all organic solvents having the capability to dissolve fats and waxes while not adversely affecting the shell polymer structure can be used. Suitable methods for defatting pollen are known in the art. To avoid that organic solvents are introduced into the alkaline solutions used in the following steps, the defatted pollen may be dried before it is subjected to the following processing steps.

The pollen or pollen extract may be subjected to a processing step, such as an intine removal step. For example, the intine can be removed by treatment with phosphoric acid or a strong base. It is believed that a strong alkali not only disrupts hydrogen bonding between hemicellulose and cellulose microfibrils or between pectin and cellulose microfibrils, but also degrades pectin and cellulose molecules along with the increased duration of alkali treatment, ultimately leading to softening of intine. The intine can then be washed away. A exine shell may be obtained as a result of this process and be used as sporopollenin in the adhesion barrier composition. The exine shell may be further purified by various washing steps, for example with water.

The degradation time of the adhesion barrier composition can be varied through the degree of degradation of sporopollenin. For example, sporopollenin can be deproteinized by contacting the pollen with an aqueous base solution at elevated temperatures for up to 10 hours. This can reduce the cross-linking of the biopolymer that makes up sporopollenin, thus allowing it to be degraded more easily under physiological conditions. As another example, sporopollenin can be hydrolytically degraded (saponification) by contacting with an aqueous base solution for periods of at least 2 days and can be up to 60 days.

Sporopollenin can undergo a deproteination step. This can be performed using a base at elevated temperatures. For example, NaOH or KOH can be used at a temperature of about 60-90° C. In some embodiments, when the sporopollenin is processed, at least 2% of the peptide bond (amide bond) is hydrolysed. In other embodiments, at least 5%, at least 10%, at least 15%, at least 20%, at least 25%, at least 30%, at least 35%, at least 40%, at least 45%, at least 50%, at least 55%, at least 60%, at least 65% or at least 70% of the peptide bond (amide bond) is hydrolysed.

The exine shell may be further processes using acids and bases. For example, alkaline conditions can be used to assist hydrolytic degradation of the sporopollenin polymer, and/or remove other components in the exine shell. The degradation can be a partial degradation such that the crosslinking density of sporopollenin is reduced relative to the naturally occurring polymer by hydrolyzing part of the crosslinking bonds, in particular ether (COC) and ester (COOC) bonds. This may, for example, be monitored by X-ray photoelectron spectroscopy (XPS). The degradation is notable by decreases in the amount of these bonds.

When the exine shell is at least partially degraded to form the sporopollenin, some of the cross-linkages (ester and acetal/ether) in sporopollenin are cleaved. The free end of the cleaved polymers are released from the surface of sporopollenin (while still being attached to sporopollenin at the uncleaved end) such that they form a “hairy” corona that further encapsulates the spikes of sporopollenin. As the corona is negatively charged due to the presence of COO⁻ and O⁻ moieties, the corona further aids in the inter-particle attraction between sporopollenin, thus allowing for the formation of a crosslinkable sporopollenin matrix (particle-particle interaction). This assists in applying sporopollenin onto a tissue surface in that slippage is minimized. The corona can also aid in the interaction between sporopollenin and biodegradable polymer, and/or between sporopollenin and tissue surface.

The at least partial degradation of the sporopollenin can be performed for up to 60 days long, typically up to 42 days, for example 7 to 35 days, 7 to 28 days, or 7 to 14 days. It is understood that by changing the process conditions, such as the temperature, shorter times may similarly be sufficient.

By varying the degradation time of the sporopollenin, the degradation time of the adhesion barrier composition can be varied from about 1 month to about 12 months.

In some embodiments, sporopollenin comprises a polymer. The polymer can have ester bonds, ether bonds, peptide bonds, or a combination thereof. The sporopollenin can also comprise fatty acids.

In various embodiments, sporopollenin is processed such that it undergoes a reduction in ester bonds during the alkaline treatment. In other embodiments, sporopollenin is processed such that it undergoes a reduction in ether bonds during the alkaline treatment. In other embodiments, sporopollenin is processed such that it undergoes a reduction in peptide bonds during the alkaline treatment. The degree of degradation can be analyzed using Attenuated total reflection-Fourier-transform infrared spectroscopy (ATR-FTIR). Peak height ratio analysis of peaks at about 1740 cm⁻¹ and about 1620 cm⁻¹ due to stretching of esterified and de-esterified carboxyl groups (—COOCH₃ and —COOH) can be used. Alternatively, a ratio can be taken relative to the most stable spectra peak. For example, the most stable peak for H. annuus is about 1062 cm⁻¹ for and attributable to a-cellulosic compounds. Alternatively, this may be monitored by X-ray photoelectron spectroscopy (XPS). The degradation is notable by decreases in the amount of these bonds.

In some embodiments, when the sporopollenin is processed, at least 2% of the ester bond is hydrolysed. In other embodiments, at least 5%, at least 10%, at least 15%, at least 20%, at least 25%, at least 30%, at least 35%, at least 40%, at least 45%, at least 50%, at least 55%, at least 60%, at least 65% or at least 70% of the ester bond is hydrolysed.

In some embodiments, when the sporopollenin is processed, at least 2% of the ether bond is hydrolysed. In other embodiments, at least 5%, at least 10%, at least 15%, at least 20%, at least 25%, at least 30%, at least 35%, at least 40%, at least 45%, at least 50%, at least 55%, at least 60%, at least 65% or at least 70% of the ether bond is hydrolysed.

In some embodiments, when the sporopollenin is processed, at least 2% of the lipid and/or fatty acid is saponificated. Saponification occurs when lipidic compounds are exposed to bases such as NaOH or KOH. This is a fundamental chemical reaction in the process of soap making, wherein triglycerides are treated with strong bases to cleave ester bonds and produce fatty acid salts and glycerol. The fatty acids can also be saponificated. This reaction involves neutralization of the carboxylic acid, and can allow for sporopollenin with more predictable physical properties. In other embodiments, at least 5%, at least 10%, at least 15%, at least 20%, at least 25%, at least 30%, at least 35%, at least 40%, at least 45%, at least 50%, at least 55%, at least 60%, at least 65% or at least 70% of the lipid and/or fatty acid is saponificated.

Without wanting to be bound by theory, it is believed that the alkaline treatment cleaves cross-linking ester and ether bonds of the sporopollenin network, reducing the cross-linking density. The alkaline treatment also does not appear to induce sizeable chemical and mechanical changes of sporopollenin backbones, preserving the inherent chain characteristics (e.g., chain stiffness, volume fraction, pKa of functional groups). Further, with the degradation process, carboxyl groups and hydroxyl groups are made available, resulting in the sporopollenin surface being covered by negative charges. A combination of charge neutralization, hydrogen bonding and ionic strength effect allows the form a sporopollenin inter-particle network such that gelation can be easily induced when required.

Accordingly, in some embodiments of a method of reducing the formation of post-surgery tissue adhesion in a subject in need thereof by administering an adhesion barrier composition, the adhesion barrier composition comprises:

a) sporopollenin, the sporopollenin comprising a polymer having ester bonds and ether bonds; and

b) a biodegradable polymer;

wherein the weight ratio of sporopollenin is about 20 wt/wt % to about 80 wt/wt % relative to the total composition;

wherein the sporopollenin is characterised with having at least 2% of the ester bond is hydrolysed; and

wherein the sporopollenin is characterised with having at least 2% of the ether bond is hydrolysed.

In some embodiments of a method of preventing the formation of tissue adhesion during or post-surgery in a subject in need thereof by administering an adhesion barrier composition, the adhesion barrier composition comprises:

a) sporopollenin, the sporopollenin comprising a polymer having ester bonds and ether bonds; and

b) a biodegradable polymer;

wherein the weight ratio of sporopollenin is about 20 wt/wt % to about 80 wt/wt % relative to the total composition;

wherein the sporopollenin is characterised with having at least 2% of the ester bond is hydrolysed; and

wherein the sporopollenin is characterised with having at least 2% of the ether bond is hydrolysed.

In some embodiments, sporopollenin comprises carboxylic moieties. In other embodiments, sporopollenin comprises phenolic moieties. In other embodiments, a mole ratio of carboxyl:phenolic moieties is about 0.5:1 to about 5:1. In other embodiments, the mole ratio is about 1:1 to about 5:1, about 1:1 to about 4:1, about 1:1 to about 3:1, or about 2:1.

Through this process, an aqueous gel can be obtained comprising about 1 to about 20 wt % sporopollenin, the remaining being the aqueous medium such as water, i.e. about 80 to about 99 wt %. In other embodiments, sporopollenin is about 1 to about 15 wt %, about 1 to about 10 wt %, about 1 to about 8 wt %, or about 1 to about 6 wt %.

In some embodiments, the sporopollenin has a shell thickness of about 5 pm to about 120 pm. In other embodiments, the thickness is about 6 to about 100 pm, about 6 to about 90 pm, or about 10 to about 100 pm. These thickness relate to the main body of the sporopollenin and do not include any sculpted surface structures, such as spikes or the like.

In some embodiments, the size of sporopollenin (particle diameter) is about 20 μm to about 50 μm. In other embodiments, the size is about 30 μm to about 50 μm, about 40 μm to about 50 μm, or about 30 μm to about 40 μm.

As used herein, ‘biodegradable polymer’ refers to a polymeric substance capable of being decomposed by bacteria or other living organisms. For example, these polymers can break down after its intended purpose by bacterial decomposition process to result in natural byproducts such as gases (CO₂, N₂), water, biomass, and inorganic salts. The polymeric substance includes biopolymers (such as DNA, peptide, protein, polysaccharides, carbohydrates) and synthetically made polymers.

In some embodiments, the biodegradable polymer is selected from poly(glycolic) acid, polylactic acid, polydioxanone, polycaprolactone, calcium alginate, sodium hyalurate, hyaluronic acid or a combination thereof. In other embodiments, the biodegradable polymer is selected from polyhydroxy butyrate (PHB), polyhydroxy butyrates-co-beta hydroxyl valerate (PHBV) or nylon-2-nylon-6. Gelatin can also be used.

In some embodiments, polylactic acid (PLA) is advantageous when used as the biodegradable polymer. As PLA can degrade into its monomer units lactic acid, like-like interactions between the carboxyl groups in the partially degraded PLA and the partially degraded sporopollenin prevent (or at least reduce) the separation (or delamination) of the adhesion barrier composition.

In some embodiments, the biodegradable polymer is provided as microspheres or nanoparticles. This allows for a homogenous dispersion to be formed and allows for an ease of application. For example, the composition can be formulated as a spray or as a gel, which solidifies on application on the tissue surface.

In some embodiments, when applied to a biological surface, the biodegradable polymer is at least 50% degraded after at least 7 days. In other embodiments, the biodegradable polymer is at least 50% degraded after at least 10 days, at least 14 days, at least 21 days, at least 28 days, at least 35 days, at least 42 days, or at least 49 days. In other embodiments, the biodegradable polymer is at least 50% degraded after less than 7 days, less than 10 days, less than 14 days, less than 21 days, less than 28 days, less than 35 days, less than 42 days, or less than 49 days.

In some embodiments, when applied to a biological surface, the biodegradable polymer is completely resorbed after at least 3 months. In other embodiments, the biodegradable polymer is completely resorbed after at least 4 months, at least 5 months, at least 6 months, at least 7 months, or at least 8 months. In other embodiments, the biodegradable polymer is completely resorbed after less than 3 months, less than 4 months, less than 5 months, less than 6 months, less than 7 months, less than 8 months, less than 12 months, less than 16 months or less than 24 months.

The amount of sporopollenin can be adjusted to facilitate the adhesion of the biological surface to the adhesion barrier composition. In this regard, it is found that if the weight ratio is too low, the adhesion barrier composition does not function properly as the composition can migrate. If the weight ratio is too high, the adhesion is aggravated. In some embodiments, the weight ratio of sporopollenin is about 1 wt/wt % to about 80 wt/wt % relative to the total composition. In other embodiments, the weight ratio of sporopollenin relative to the total composition is about 1 wt/wt % to about 70 wt/wt %, about 1 wt/wt % to about 60 wt/wt %, about 1 wt/wt % to about 50 wt/wt %, about 1 wt/wt % to about 40 wt/wt %, about 1 wt/wt % to about 30 wt/wt %, or about 1 wt/wt % to about 20 wt/wt %. In other embodiments, the weight ratio of sporopollenin relative to the total composition is about 20 wt/wt % to about 80 wt/wt %, about 20 wt/wt % to about 70 wt/wt %, or about 20 wt/wt % to about 60 wt/wt %. For example, when a film is formed and coated on one side with sporopollenin, the weight ratio is about 20 wt/wt % to about 50 wt/wt %, about 25 wt/wt % to about 50 wt/wt %, about 30 wt/wt % to about 50 wt/wt %, about 20 wt/wt % to about 45 wt/wt %, about 20 wt/wt % to about 40 wt/wt %, about 25 wt/wt % to about 40 wt/wt %, about 30 wt/wt % to about 40 wt/wt %, or about 35 wt/wt % to about 40 wt/wt %. For example, when a film is formed and coated on both side with sporopollenin, the weight ratio is about 40 wt/wt % to about 80 wt/wt %, about 40 wt/wt % to about 75 wt/wt %, about 40 wt/wt % to about 70 wt/wt %, about 40 wt/wt % to about 65 wt/wt %, about 40 wt/wt % to about 60 wt/wt %, about 45 wt/wt % to about 80 wt/wt %, about 50 wt/wt % to about 80 wt/wt %, about 50 wt/wt % to about 75 wt/wt %, about 50 wt/wt % to about 70 wt/wt %, about 50 wt/wt % to about 65 wt/wt %, about 50 wt/wt % to about 60 wt/wt %, or about 50 wt/wt % to about 55 wt/wt %.

In some embodiments, the adhesion barrier composition further comprises a solvent. In some embodiments, the solvent is an aqueous medium. The term ‘aqueous medium’ used herein refers to a water based solvent or solvent system, and which comprises of mainly water. Such solvents can be either polar or non-polar, and/or either protic or aprotic. Solvent systems refer to combinations of solvents which resulting in a final single phase. Both ‘solvents’ and ‘solvent systems’ can include, and is not limited to, pentane, cyclopentane, hexane, cyclohexane, benzene, toluene, dioxane, chloroform, diethylether, dichloromethane, tetrahydrofuran, ethyl acetate, acetone, dimethylformamide, acetonitrile, dimethyl sulfoxide, nitromethane, propylene carbonate, formic acid, butanol, isopropanol, propanol, ethanol, methanol, acetic acid, ethylene glycol, diethylene glycol or water. Water based solvent or solvent systems can also include dissolved ions, salts and molecules such as amino acids, proteins, sugars and phospholipids. Such salts may be, but not limited to, sodium chloride, potassium chloride, ammonium acetate, magnesium acetate, magnesium chloride, magnesium sulfate, potassium acetate, potassium chloride, sodium acetate, sodium citrate, zinc chloride, HEPES sodium, calcium chloride, ferric nitrate, sodium bicarbonate, potassium phosphate and sodium phosphate. As such, biological fluids, physiological solutions and culture medium also falls within this definition.

The solvent can improve the dispersion of sporopollenin in the composition and can aid its application on a surface. The solvent also allows the adhesion barrier composition to be maintained in an applicable state for use.

In some embodiments, the weight ratio of the solvent is at least 2 wt/wt % relative to the total composition. In other embodiments, the weight ratio of the solvent relative to the total composition is at least 5 wt/wt %, at least 10 wt/wt %, at least 15 wt/wt %, at least 20 wt/wt %, at least 25 wt/wt %, at least 30 wt/wt %, at least 35 wt/wt %, at least 40 wt/wt %, at least 45 wt/wt %, at least 50 wt/wt %, at least 55 wt/wt %, at least 60 wt/wt %, at least 65 wt/wt % or at least 70 wt/wt %.

In some embodiments, the adhesion barrier composition further comprises a lipid. The lipid can be a hydrophobic lipid or amphiphilic lipid. For example, the lipid can be a fatty acid, glycerolipid, glycerophospholipid, sphingolipid, saccharolipid, polyketides, sterol lipids and prenol lipids. Lipids also encompasses its derivatives (including tri-, di-, monoglycerides, and phospholipids) as well as other sterol-containing metabolites such as cholesterol.

The lipid can improve the dispersion of sporopollenin in the composition. The lipid also increases the bioadhesion of sporopollenin and hence the composition to the surface. This is believed to be due to the formation of capillary bridges, which acts in combination with the convex, spiny surfaces of sporopollenin to enhance the wettability and hence spreading area of sporopollenin and accordingly the composition. The adhesive strength of capillary bridges is believed to be dependent on non-polar van der Waals interactions, with some contribution from the Lewis base component of surface energy.

In some embodiments, the lipid is selected from lauric acid, palmitic acid, α-linolenic acid, myristic acid, stearic acid, oleic acid, linoleic acid, eicosenoic acid, glycerides or a combination thereof.

In some embodiments, the weight ratio of the lipid is at least 2 wt/wt % relative to the total composition. In other embodiments, the weight ratio of the lipid relative to the total composition is at least 3 wt/wt %, at least 4 wt/wt %, at least 5 wt/wt %, at least 6 wt/wt %, at least 7 wt/wt %, at least 8 wt/wt %, at least 9 wt/wt %, at least 10 wt/wt %, at least 15 wt/wt %, at least 20 wt/wt %, at least 25 wt/wt %, at least 30 wt/wt %, at least 35 wt/wt % or at least 40 wt/wt %.

In some embodiments, the adhesion barrier composition further comprises pollenkitt. Pollenkitt is an adhesive material present around pollen grains of angiosperms. During the process of purifying/extracting sporopollenin, pollenkitt can be extracted as a component. This component can be subsequently added to the adhesion barrier composition to further improve the adhesion function of the composition. As pollenkitt is a biomaterial, it is expected to be safe and non-toxic to animals.

In some embodiments, the pollenkitt is selected from an angiosperm. Angiosperm is a flowering, fruit-bearing plant or tree known for having ovules (and therefore seeds) develop within an enclosed ovary. Angiosperms are plants producing flowers.

In some embodiments, the angiosperm is selected from the genus Baccharis, Helianthus or Camellia, Coreopsis, Callistephus, Dahalia, Solidago or a combination thereof.

In some embodiments, the weight ratio of the pollenkitt is at least 2 wt/wt % relative to the total composition. In other embodiments, the weight ratio of the pollenkitt relative to the total composition is at least 3 wt/wt %, at least 4 wt/wt %, at least 5 wt/wt %, at least 6 wt/wt %, at least 7 wt/wt %, at least 8 wt/wt %, at least 9 wt/wt %, at least 10 wt/wt %, at least 15 wt/wt %, at least 20 wt/wt %, at least 25 wt/wt %, at least 30 wt/wt %, at least 35 wt/wt % or at least 40 wt/wt %.

In some embodiments, the adhesive barrier composition further comprises intine. In other embodiments, the adhesive barrier composition further comprises an intine material. The intine is the inner layer of a pollen. It was found that the intine extends the degradation time, and thus allows the adhesive barrier composition to be fine tuned for various applications.

In some embodiments, the intine is selected from polysaccharides, cellulose, hemicellulose, pectin, or a combination thereof. The intine can be at least partially degraded, in a process similar to that with sporopollenin.

In some embodiments, the adhesion barrier composition further comprises phenolic compound. These are chemical compounds which consisting of a hydroxyl group bonded directly to an aromatic hydrocarbon group. As used herein, ‘phenolic compounds’ also include macromolecules and polymers having phenol moieties or side chains.

The phenolic compound can attenuate postoperative adhesion. The degree of inflammation and fibrosis can also be reduced. It is believed that phenolic compounds can attenuate the cellular mechanism and signalling pathway of fibrogenesis that leads to postoperative adhesion formation. For example, gallic acid can decrease intra-abdominal adhesions in rats as compared to a control. Further studies have shown that IL-6, TNF-α, TGF-b1 serum levels and NF-kB phosphorylation were significantly reduced.

In some embodiments, the phenolic compound is selected from gallic acid, caffeic acid, ferulic acid, p-coumaric, benzoic acid or a combination thereof.

In some embodiments, the weight ratio of the phenolic compound is at least 1 wt/wt % relative to the total composition. In other embodiments, the weight ratio of the phenolic compound relative to the total composition is at least 2 wt/wt %, at least 3 wt/wt %, at least 4 wt/wt %, at least 5 wt/wt %, at least 6 wt/wt %, at least 7 wt/wt %, at least 8 wt/wt %, at least 9 wt/wt %, at least 10 wt/wt %, at least 20 wt/wt %, at least 25 wt/wt %, at least 30 wt/wt %, at least 35 wt/wt % or at least 40 wt/wt %.

Other additives such as gelatin and sodium caboxymethy cellulose (CMC) can be incorporated in the composition to fine-tune the material properties of the film such as mechanical strength, flexibility, and to achieve hydrogel properties of the sporopollenin film. The additive can be added at less than about 20 wt %, about 15 wt %, about 10 wt %, about 9 wt %, about 8 wt %, about 7 wt %, about 6 wt %, about 5 wt %, about 4 wt %, about 3 wt %, about 2 wt %, or about 1 wt %.

Accordingly, in some embodiments, the adhesion barrier composition comprises:

a) sporopollenin; and

b) a biodegradable polymer;

wherein the weight ratio of sporopollenin is about 1 wt/wt % to about 80 wt/wt % relative to the total composition and;

wherein the sporopollenin is processed such that at least 2% of peptide bonds is hydrolysed.

In some embodiments, the adhesion barrier composition comprises:

a) sporopollenin, the sporopollenin comprising a polymer having peptide bonds; and

b) a biodegradable polymer;

wherein the weight ratio of sporopollenin is about 20 wt/wt % to about 80 wt/wt % relative to the total composition and;

wherein the sporopollenin is processed such that at least 2% of peptide bonds is hydrolysed.

In other embodiments, the adhesion barrier composition comprises:

a) sporopollenin;

b) a biodegradable polymer; and

c) a phenolic compound;

wherein the weight ratio of sporopollenin is about 1 wt/wt % to about 80 wt/wt % relative to the total composition and;

wherein the sporopollenin is processed such that at least 2% of peptide bonds is hydrolysed;

wherein the phenolic compound is selected from gallic acid, caffeic acid, ferulic acid, p-coumaric phenolic acid, benzoic acid or a combination thereof; and

the weight ratio of the phenolic compound is at least 1 wt/wt % relative to the total composition.

In other embodiments, the adhesion barrier composition comprises:

a) sporopollenin, the sporopollenin comprising a polymer having peptide bonds;

b) a biodegradable polymer; and

c) a phenolic compound;

wherein the weight ratio of sporopollenin is about 20 wt/wt % to about 80 wt/wt % relative to the total composition and;

wherein the sporopollenin is processed such that at least 2% of peptide bonds is hydrolysed;

wherein the phenolic compound is selected from gallic acid, caffeic acid, ferulic acid, p-coumaric phenolic acid, benzoic acid or a combination thereof; and the weight ratio of the phenolic compound is at least 1 wt/wt % relative to the total composition.

In some embodiments, the adhesion barrier composition comprises:

a) sporopollenin, the sporopollenin comprising a polymer having ester bonds and ether bonds; and

b) a biodegradable polymer;

wherein the weight ratio of sporopollenin is about 20 wt/wt % to about 80 wt/wt % relative to the total composition;

wherein the sporopollenin is processed such that at least 2% of the ester bond is hydrolysed; and wherein the sporopollenin is processed such that at least 2% of the ether bond is hydrolysed.

In other embodiments, the adhesion barrier composition comprises:

a) sporopollenin, the sporopollenin comprising a polymer having ester bonds and ether bonds;

b) a biodegradable polymer; and

c) a phenolic compound;

wherein the weight ratio of sporopollenin is about 20 wt/wt % to about 80 wt/wt % relative to the total composition;

wherein the sporopollenin is processed such that at least 2% of the ester bond is hydrolysed;

wherein the sporopollenin is processed such that at least 2% of the ether bond is hydrolysed;

wherein the phenolic compound is selected from gallic acid, caffeic acid, ferulic acid, p-coumaric phenolic acid, benzoic acid or a combination thereof; and

the weight ratio of the phenolic compound is at least 1 wt/wt % relative to the total composition.

In some embodiments, when applied to a biological surface, the adhesion barrier composition is at least 50% degraded after at least 7 days. In other embodiments, the adhesion barrier composition is at least 50% degraded after at least 10 days, at least 14 days, at least 21 days, at least 28 days, at least 35 days, at least 42 days, or at least 49 days.

In some embodiments, when applied to a biological surface, the adhesion barrier composition is completely resorbed after at least 3 months. In other embodiments, the adhesion barrier composition is completely resorbed after at least 4 months, at least 5 months, at least 6 months, at least 7 months, at least 8 months, at least 12 months, at least 16 months or at least 24 months. In other embodiments, the adhesion barrier composition is completely resorbed after less than 3 months, less than 4 months, less than 5 months, less than 6 months, less than 7 months, less than 8 months, less than 12 months, less than 16 months or less than 24 months.

In some embodiments, the adhesion barrier composition has a transparency of at least 90%. In other embodiments, the adhesion barrier composition has a transparency of at least 91%, at least 92%, at least 93%, at least 94%, at least 95%, at least 96%, at least 97%, at least 98%, or at least 99%.

In some embodiments, the adhesion barrier composition is a film, gel or a liquid.

When formed as a film, the film can have a thickness of about 0.1 μm. In other embodiments, the thickness is about 0.5 μm, about 1 μm, about 5 μm, about 10 μm, about 20 μm, about 50 μm, about 100 μm, about 200 μm, about 500 μm, about 700 μm, or about 1 mm. The film can be a layered film comprising alternating layers of sporopollenin and biodegradable polymer, or can be a homogenous film in which sporopollenin and the biodegradable polymer are homogenously mixed or blended.

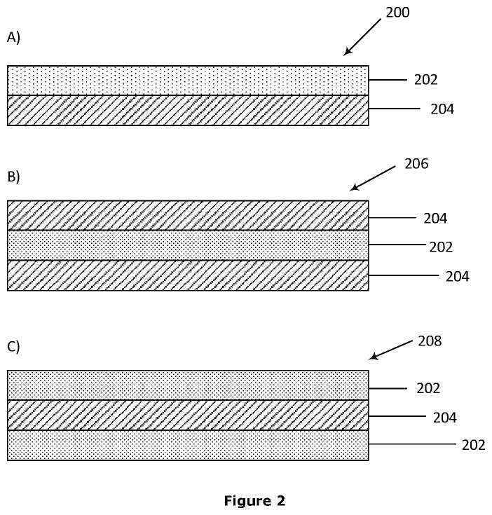

In some embodiments, when formed as a single layered film, the film comprises sporopollenin and biodegradable polymer homogenously mixed or blended. In other embodiments, when formed as a multilayered film, the adhesive barrier composition comprises a layer of sporopollenin and a layer of biodegradable polymer film (FIG. 2a). The layer of sporopollenin is adjacent and in contact with the layer of biodegradable polymer. In other embodiments, the layer of sporopollenin is sandwiched between two layers of biodegradable polymers (FIG. 2b). In other embodiments, the layer of biodegradable polymer is sandwiched between two layers of sporopollenin (FIG. 2c). The layer of sporopollenin can comprise sporopollenin and optionally a biodegradable polymer. For example, the layer of sporopollenin can comprise sporopollenin and polylactic acid. When layered with the layer of biodegradable polymer, the layer of biodegradable polymer can be the same biodegradable polymer used in the layer of sporopollenin, or a different biodegradable polymer.

In some embodiments, when the adhesion barrier composition is an adhesion barrier film, sporopollenin is formed as a coating on the biodegradable polymer; i.e. coats a surface of the biodegradable polymer. The sporopollenin can be coated on the biodegradable polymer as a monolayered coating. In some embodiments, when an adhesion barrier film is formed, the coating is such that 100% of a surface of the biodegradable polymer is covered. In this regard, sporopollenin covers 100% of the surface. This can, for example, be characterised using microscopy techniques such as scanning electron microscopy. In other embodiments, the coating is about 90%, about 80%, about 70%, about 60%, about 50%, or about 40%.

In some embodiments, sporopollenin has a surface coverage on a surface of the biodegradable polymer of about 10% to about 99%. The surface coverage can, for example, be estimated using microscopy techniques and image analysis software. In other embodiments, the surface coverage is about 15% to about 99%, about 20% to about 99%, about 25% to about 99%, about 30% to about 99%, about 35% to about 99%, about 40% to about 99%, about 45% to about 99%, about 50% to about 99%, about 55% to about 99%, about 60% to about 99%, about 65% to about 99%, about 70% to about 99%, about 75% to about 99%, or about 80% to about 99%.

In some embodiments, when a monolayer of sporopollenin layer is formed, it has a thickness of about 30 μm to about 60 μm. In other embodiments, the thickness is about 30 μm to about 55 μm, about 35 μm to about 55 μm, about 40 μm to about 55 μm, or about 40 μm to about 50 μm.

In other embodiments, the sporopollenin forms a multilayered coating on the biodegradable polymer. The coating can have a thickness of about 50 μm to about 200 μm. In other embodiments, the thickness is about 50 μm to about 180 μm, about 50 μm to about 170 μm, about 50 μm to about 160 μm, about 50 μm to about 150 μm, about 50 μm to about 140 μm, about 50 μm to about 130 μm, about 50 μm to about 120 μm, about 50 μm to about 110 μm, about 50 μm to about 100 μm, about 50 μm to about 90 μm, or about 50 μm to about 80 μm.

In some embodiments, when the adhesion barrier composition is an adhesion barrier film, the biodegradable polymer has a thickness of about 1 μm to about 200 μm. In other embodiments, the thickness is about 1 μm to about 40 μm, about 1 μm to about 30 μm, about 1 μm to about 20 μm, about 1 μm to about 10 μm, or about 5 μm to about 10 μm. In other embodiments, the thickness is about 10 μm to about 50 μm, about 20 μm to about 50 μm, about 30 μm to about 50 μm, or about 40 μm to about 50 μm. In other embodiments, the thickness is about 10 μm to about 200 μm, about 10 μm to about 180 μm, about 10 μm to about 160 μm, about 10 μm to about 140 μm, about 10 μm to about 120 μm, about 10 μm to about 100 μm, about 10 μm to about 80 μm, or about 10 μm to about 60 μm.

In some embodiments, when the adhesion barrier composition is an adhesion barrier film, the adhesion barrier film comprises:

a) a coating of sporopollenin; and

b) a biodegradable polymer film having two surfaces;

wherein the weight ratio of sporopollenin is about 1 wt/wt % to about 80 wt/wt % relative to the total composition; and

wherein sporopollenin coats at least one surface of the biodegradable polymer film.

In some embodiments, when the adhesion barrier composition is an adhesion barrier film, the adhesion barrier film comprises:

a) a coating of sporopollenin, the sporopollenin comprising a polymer having ester bonds and ether bonds; and

b) a biodegradable polymer film having two surfaces;

wherein the weight ratio of sporopollenin is about 20 wt/wt % to about 80 wt/wt % relative to the total composition;

wherein the sporopollenin is processed such that at least 2% of the ester bond is hydrolysed;

wherein the sporopollenin is processed such that at least 2% of the ether bond is hydrolysed; and

wherein sporopollenin coats at least one surface of the biodegradable polymer film.

When formed as a gel or liquid, the adhesion barrier composition can be in a spray-able or injectable form via a syringe. In other embodiments, the adhesion barrier composition has a viscosity of about 0.005 Pa·s⁻¹ to about 20 Pa·s⁻¹. In other embodiments, the viscosity is about 0.01 Pa·s⁻¹ to about 20 Pa·s⁻¹, about 0.015 Pa·s⁻¹ to about 20 Pa·s⁻¹, about 0.1 Pa·s⁻¹ to about 20 Pa·s⁻¹, about 0.15 Pa·s⁻¹ to about 20 Pa·s⁻¹, about 0.5 Pa·s⁻¹ to about 20 Pa·s⁻¹, about 1 Pa·s⁻¹ to about 20 Pa·s⁻¹, about 1 Pa·s⁻¹ to about 18 Pa·s⁻¹, about 1 Pa·s⁻¹ to about 15 Pa·s⁻¹, or about 5 Pa·s⁻¹ to about 15 Pa·s⁻¹.

Examples of different types of gels that falls within the scope of this invention are for example, hydrogel, oranogel, xerogel and nanocomposite hydrogel.

The adhesion barrier compositions suitable for administration to the biological surface may further comprise any suitable carrier or base and may be in the form of lotions, gel, creams, pastes, ointments and the like. Suitable carriers include mineral oil, propylene glycol, polyoxyethylene, polyoxypropylene, emulsifying wax, sorbitan monostearate, polysorbate 60, cetyl esters wax, cetearyl alcohol, 2-octyldodecanol and benzyl alcohol.

The adhesion barrier composition may contain any suitable carriers, diluents or excipients. These include all conventional solvents, dispersion media, fillers, solid carriers, coatings, antifungal and antibacterial agents, surfactants, isotonic and absorption agents and the like. It will be understood that the compositions of the invention may also include other supplementary physiologically active agents.

The present invention also relates to a method of fabricating an adhesion barrier composition, comprising:

a) dispersing a sporopollenin in or on a biodegradable polymer;

wherein the weight ratio of sporopollenin is about 1 wt/wt % to about 80 wt/wt % relative to the total composition.

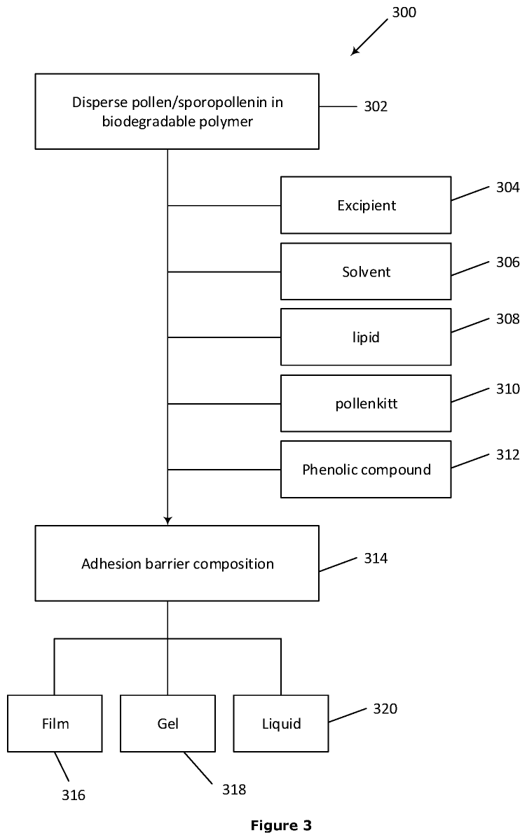

For example, the sporopollenin can be dispersed in the biodegradable polymer via vortexing (FIG. 3; 302). Addition of other excipients (304) may also be added to facilitate the dispersion of sporopollenin in the biodegradable polymer.

In some embodiments, the method further comprises a step of dispersing sporopollenin and the biodegradable polymer in a solvent (306).

In other embodiments, the method further comprises a step of dispersing a lipid (308) in the biodegradable polymer.

In other embodiments, the method further comprises a step of dispersing a pollenkitt (310) in the biodegradable polymer.

In other embodiments, the method further comprises a step of dispersing a phenolic compound (312) in the biodegradable polymer.

In other embodiments, the method further comprises a step of forming the adhesion barrier composition (314) as a film (316), gel (318) or liquid (320).

In some embodiments, the method of fabricating an adhesion barrier composition comprises:

a) dispersing a sporopollenin in or on a biodegradable polymer;

wherein the weight ratio of sporopollenin is about 20 wt/wt % to about 80 wt/wt % relative to the total composition;

wherein the sporopollenin comprising a polymer having ester bonds and ether bonds;

wherein the sporopollenin is processed such that at least 2% of the ester bond is hydrolysed; and

wherein the sporopollenin is processed such that at least 2% of the ether bond is hydrolysed.

In some embodiments, when forming the adhesion barrier composition as a film, the method of fabrication comprises:

a) dispersing a coating of sporopollenin on a biodegradable polymer film;

wherein the weight ratio of sporopollenin is about 1 wt/wt % to about 80 wt/wt % relative to the total composition.

In some embodiments, when forming the adhesion barrier composition as a film, the method of fabrication comprises:

a) dispersing a coating of sporopollenin on a biodegradable polymer film;

wherein the weight ratio of sporopollenin is about 20 wt/wt % to about 80 wt/wt % relative to the total composition;

wherein the sporopollenin comprising a polymer having ester bonds and ether bonds;

wherein the sporopollenin is processed such that at least 2% of the ester bond is hydrolysed; and

wherein the sporopollenin is processed such that at least 2% of the ether bond is hydrolysed.

In an alternative method, when forming the adhesion barrier composition as a film, the method of fabrication can comprise:

a) dispersing a sporopollenin in an aqueous solvent to form a sporopollenin dispersion;

b) drying the sporopollenin dispersion to form a sporopollenin layer; and

c) contacting the sporopollenin layer on a surface of a biodegradable polymer film to form the adhesion barrier film;

wherein the weight ratio of sporopollenin is about 20 wt/wt % to about 80 wt/wt % relative to the total composition;

wherein the sporopollenin comprising a polymer having ester bonds and ether bonds;

wherein the sporopollenin is processed such that at least 2% of the ester bond is hydrolysed; and

wherein the sporopollenin is processed such that at least 2% of the ether bond is hydrolysed.

The sporopollenin dispersion can further comprise a biodegradable polymer. Other excipients may also be added to facilitate the dispersion of sporopollenin.

The method can further comprise a step of contacting a second sporopollenin layer on an other surface of the biodegradable polymer, such that the biodegradable polymer is sandwiched between two layers of sporopollenin. Alternatively, the method can further comprise a step of contacting a second biodegradable polymer on another surface of the sporopollenin layer, such that the sporopollenin layer is sandwiched between two layers of biodegradable polymer.

The adhesion barrier composition as disclosed herein is suitable for use in preventing adhesion. As mentioned above, adhesion can occur from surgery following wound healing, and accordingly can be suitable for use in gynaecological surgery, general surgery, spine surgery and cardiac surgery.

### Examples

Pollen of Helianthus Annuus is obtained from Greer Labs (USA). The pollen has a grain size of 45.2 μm, and the as supplied pollen has a 0.8% plant parts (anemorphilous plants and entomophilous plants) and 0.5% contamination (foreign pollen, mold spores, smut spores, rust spores). The moisture content is less than 3%.

Fabrication of sporopollenin: Firstly, a 10 vol % food grade NaOH/water solution is prepared. A 250 ml of the NaOH solution will have 5 g of the Helianthus Annuus pollen, used as supplied, added to the solution. The solution was heated in a water bath maintained at 80° C. for 2 hours with constant magnetic stirring of the pollen-NaOH solution. The solution was left at room temperature for 24 hours and centrifuged a few times at 4000 rpm to remove the supernatant liquid. A fresh 10 vol % food grade NaOH/water solution is added to the remaining microgel of sporopollenin. The mixture was ultrasonically treated and magnetic stirred for 1 hour and left at room temperature for a week. Mixture was washed multiple times by centrifuge using deionised water until the remaining microgel was at pH 8.0-8.5.

Fabrication of gel (318): The gel can be prepared by vortexing (2 min) the sporopollenin in KOH (aq) as a suspension to ensure uniform mixing, reflux for 22 hrs at 80° C. and aliquoting the suspension after cooling into a 15 ml falcon tube for water washing. The aliquoted suspension was topped up to 10 ml with distilled water and vortexed at high speed (2 min), followed by centrifugation (4500 rpm, 5 min). The supernatant was removed and measured for pH. If the supernatant pH was greater than 7.5, the tube was topped up to 10 ml with distilled water, followed again by vortexing and centrifugation. The water washing was typically repeated for a total of 4 to 6 washes to achieve neutral pH (˜7.5). This sporopollenin was transferred to a 2 ml vial using a 3 ml disposable dropper, and topped up to 2 ml with distilled water and an appropriate amount of biodegradable polymer such as microspheres or nanoparticles of poly(glycolic) acid and vortexed briefly to mix. The suspension was centrifuged (14000 rpm, 5 min) and the supernatant was removed. The centrifugation and supernatant removal process was repeated for a total of 3 times, so as to remove as much free water as possible.

Fabrication of single layered film (316): Film was prepared by vortexing (2 min) sporopollenin in KOH (aq) as a suspension to ensure uniform mixing, reflux for 22 hrs at 80° C., aliquoting the suspension after cooling into a 50 ml falcon tube, centrifugation (4500 rpm, 5 min) with removal of supernatant, then addition of warm water (20 ml, 50° C.), vortexing, transfer to a glass beaker, and the addition of more warm water (130 ml, 50° C.). The suspension was stirred (5 min) and then the water was removed via vacuum filtration using a nylon mesh (10 miti) as the filter. The sporopollenin was recovered into a glass beaker and topped up to 150 ml with water followed by stirring (5 min). The washing and vacuum filtration steps were repeated until the aqueous sporopollenin suspension reached neutral pH (˜7.5). After the final filtration step, an appropriate amount of biodegradable polymer such as in the form of microspheres, or in the case of polylactic acid dissolved in chloroform, was added to the sporopollenin and mixed homogenously. The appropriate amount of the mixture was transferred from the stock mixture into a large surface area glass petri dish. The mixture was allowed to dry in the petri dish by running the vacuum pump (10 min), and convection drying in a fume hood (48 h) and further dried by exposure to a low vacuum environment (10 mbar) until stable weight (˜6 h). A continuous film will be formed after complete dryness when all aqueous solvent and organic solvent such as chloroform were fully evaporated.

Fabrication of multilayered film: Film was prepared by vortexing (2 min) sporopollenin in KOH_{aq} as a suspension to ensure uniform mixing, reflux for 2 hrs at 80° C., aliquoting the suspension after cooling into a 50 ml falcon tube, centrifugation (4500 rpm, 5 min) with removal of supernatant, then addition of warm water (20 ml, 50° C.), vortexing, transfer to a glass beaker, and the addition of more warm water (130 ml, 50° C.). The suspension was stirred (5 min) and then the water was removed via vacuum filtration using a nylon mesh (10 miti) as the filter. The sporopollenin was recovered into a glass beaker and topped up to 150 ml with water followed by stirring (5 min). The washing and vacuum filtration steps were repeated until the aqueous sporopollenin suspension reached neutral pH (˜7.5). An appropriate amount of the mixture was transferred onto a biodegradable film (204) placed in a petri dish. The mixture was allowed to dry in the petri dish by running the vacuum pump (10 min), and convection drying in a fume hood (48 h) and further dried by exposure to a low vacuum environment (10 mbar) until stable weight (˜6 h). A continuous film (202) will be formed on the biodegradable film (204) rendering a 2 layered film (FIG. 2A; 200) after complete dryness when all solvent was evaporated.

Subsequent biodegradable film (204) could be added on top of the sporopollenin film to produce a 3 layered film (FIG. 2B; 206), or conversely, sporopollenin films could be formed in contact and adjacent to both planar surfaces of the biodegradable film (204) to produce an alternative 3 layered film (FIG. 2C; 208) by the same process described above.

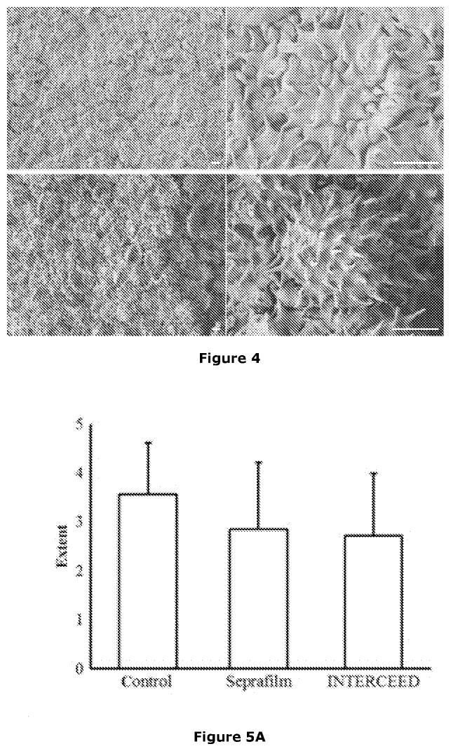

For example, the sporopollenin microgel can be solution-coated on a plastic moulding tray with the PLA film at the base. The set-up is left at room temperature to dry after which the other surface of the PLA film could be similarly solution-coated by the sporopollenin microgel. Using the above method, an adhesion barrier film can comprise a 15 cm×5.5 cm PLA film (weight about 0.6 g). Both surfaces of the PLA film can be coated with the sporopollenin, to give a final weight of about 1.3 g. The PLA film is 50 μm, while each sporopollenin layer on each PLA surface is about 50-100 μm. The coating is uniform such that 100% coverage is achieved on both surfaces. The SEM surface morphology of the sporopollenin film is shown in FIG. 4 (scale bar is 10 μm).

In-vivo test general protocol: The adhesion barrier compositions can be tested in vivo in any animal models suitable for testing. For example, adhesion barrier films can be tested in rats. The general experimental protocol is given below.

The animals (n=20) were numbered in a random matter (1-20) and anesthetized by an intramuscular injection of 60 mg/kg ketamine and 16 mg/kg xylazine. After shaving and disinfecting the abdomen, a 60 mm long midline laparotomy was performed to gain access to the abdominal cavity. Standardized peritoneal trauma was inflicted to both lateral peritoneal sides. A 15 mm long sharp incision was made through the musculoperitoneal tissue on the left lateral abdominal wall just above the epigastric vessel parallel with the midline incision. The injury site was immediately closed with four interrupted sutures placed equidistantly using 4-0 polypropylene threads (Prolene; Ethicon, Johnson & Johnson). On the right lateral side 10 mm² peritoneum including a part of the underlying muscle was excised creating a traumatized area. Before closing the abdomen, randomly chosen animals (n=10) were treated with adhesion barrier film as reported in the following, respectively. Non-treated animals (n=10) were used as control.

The two groups are as follows:

1. Adhesion barrier film (n=10): Film of 30 mm×30 mm is placed directly over the sites of injury.

2. Control (n=10): No anti-adhesive treatment was given.

The midline laparotomy was then closed in two layers with 4-0 polypropylene sutures and Vetbond. The animals were kept on a heating pad during surgery until recovery from anesthesia and thereafter returned to their respective cages. 21 days (3 weeks) after the surgery, the animals were sacrificed and relaparotomized to check for internal adhesions at the sites of injury. This was done to analyse the macro and microscopic aspects of the possible postoperative adhesions and test the tolerance of the product after three weeks of implantation. Macroscopic observation adhesions can classified into four classes (based on visual observations and photographs):

Class 0: Total lack of adhesion

Class I: minimal adhesion, peel finger

Class II: moderate adhesion. Dissection is possible with scissors and the cleavage plane is easy to spot

Class III: severe adhesion. No cleavage plane.

Microscopic observation is intended to mark the possibility of inflammatory reaction and the detection of the occurrence of adhesion to the body. The observation was made firstly in the product absorption and then after total digestion of the biomaterial.

Animal study on adhesion barrier: The adhesion prevention efficacy of the adhesion barrier film was evaluated using a rat model of sidewall defect-cecum abrasion. The cecum and adjacent tissue of the abdominal wall of Sprague Dawley rats were traumatized during surgery according to procedures described in the IACUC Animal Protocol #62020-53. After 21 days rats were sacrificed and adhesions at the site of injury were quantitated semi-quantitatively as described in the next section.

Materials Required:
Animals: 20 adult male inbred rats weighing approximately 200 g, 10 for control and 10 for in-vivo procedure
Anaesthetics for mouse eg. chloral hydrate (10%)
Sutures (Prolene, Ethicon, Johnson & Johnson)
Adhesion barrier film: 30 mm×30 mm

Experimental Procedure In Vivo:

The animals (n=20) were numbered in a random matter (1-20) and anesthetized with chloral hydrate (10%, 3 mL/kg via intraperitoneal injection). After shaving and disinfecting the abdomen with alcohol and iodine solution, a 40 mm long midline laparotomy was performed along the linea alba to gain access to the abdominal cavity. A 20 mm2 defect of the cecum with oozing blood was developed by abraded with surgical gauze. Meanwhile, a 20 mm by 20 mm apposing parietal peritoneal defect with punctate haemorrhage was created using scalpel in the right abdominal wall and the wood surfaces were gently dabbed with sterile gauze to remove excess pooled blood. Before closing the abdomen, randomly chosen animals (n=10) were treated with adhesion barrier film as reported in the following, respectively. Non-treated animals (n=10) were used as control.

1. Adhesion barrier film (n=10): Film of 30 mm×30 mm is placed directly over the sites of injury.

2. Control (n=10): No anti-adhesive treatment was given.

The two injured surfaces were approximated with 3-0 silk suture. The peritoneum was closed with 3-0 silk sutures, and the skin was closed with 4-0 silk sutures. The animals were kept on a heating pad during surgery until recovery from anaesthesia and thereafter returned to their respective cages.

Evaluation of Post-Surgery Intraperitoneal Adhesions:

21 days (3 weeks) after the surgery, the animals were sacrificed and relaparotomized to check for internal adhesions at the sites of injury. Photographs to be taken for both group of animals: Biobarrier (n=10) and Control (n=10) at the sites of injury.

Each rat was evaluated according to the following standard adhesion scoring system which has been widely used in this field, for example based on Lin L-X, et. al. (2017) PLoS ONE 12(2): e0172088.

TABLE 1
Adhesion Severity and Adhesion Area Scoring Scheme.

| | Description | |
|---|---|---|
| Degree | Adhesion Severity | Adhesion Area |
| 0 | No adhesions | No adhesions |
| 1 | Thin filmy adhesion | ≤25% of initial injured area |
| 2 | More than one thin adhesion | 25-50% of initial injured area |
| 3 | Thick adhesion with focal point | 50-70% of initial injured area |
| 4 | Thick adhesion with planar attachment | 75-100% of initial injured area |

T- Test Statistical Analysis

| | Animal | | |
|---|---|---|---|
| Group | No. | Scoring | Remarks |
| Control | 1 | 4 | A thick planar adhesion between traumatized |
| | | | areas of the cecum and abdominal wall was |
| | | | observed. No other adhesions were found. |
| | 2 | 4 | A thick planar adhesion between traumatized |
| | | | areas of the cecum and abdominal wall was |
| | | | observed. No other adhesions were found. |
| | 3 | 4 | A thick planar adhesion between traumatized |
| | | | areas of the cecum and abdominal wall was |
| | | | observed. No other adhesions were found. |
| | 4 | 4 | A thick planar adhesion between traumatized |
| | | | areas of the cecum and abdominal wall was |
| | | | observed. No other adhesions were found. |
| | 5 | 4 | A thick planar adhesion between traumatized |
| | | | areas of the cecum and abdominal wall was |
| | | | observed. No other adhesions were found. |
| | 11 | 4 | A thick planar adhesion between traumatized |
| | | | areas of the cecum and abdominal wall was |
| | | | observed. No other adhesions were found. |
| | 12 | 4 | A thick planar adhesion between traumatized |
| | | | areas of the cecum and abdominal wall was |
| | | | observed. No other adhesions were found. |
| | 13 | 4 | A thick planar adhesion between traumatized |
| | | | areas of the cecum and abdominal wall was |
| | | | observed. No other adhesions were found. |
| | 14 | NA | Died post-surgery |
| | 15 | NA | Died post-surgery |
| | Mean | 4 | Average |
| | SD | 0.0 | Standard Deviation |
| | SEM | 0.0 | Standard Error of the Mean |
| Treatment | 6 | 0 | No cecum-abdomen adhesion was found. The |
| | | | sheet had moved to the upper left side of the |
| | | | abdomen and partially overlapped with liver. |
| | 7 | 2 | A light yellow coloured sphere was found |
| | | | between the abdominal wall and the bottom of |
| | | | the cecum, making spot connections in |
| | | | between. The ball turned out to be the wrinkled |
| | | | sheet surrounded by adipose tissue. |
| | 8 | 0 | No cecum-abdomen adhesion was found. The |
| | | | sheet had moved up to up to the left side of the |
| | | | abdomen, and had partial overlap with the |
| | | | liver. |
| | 9 | 0 | No cecum-abdomen adhesion was found. |
| | 10 | 0 | A light yellow coloured sphere was found |
| | | | between the abdominal wall and the side of the |
| | | | cecum, making spot connections in between. |
| | | | The ball turned out to be the wrinkled sheet |
| | | | surrounded by adipose tissue. |
| | 16 | 2 | A light yellow coloured pocket was found |
| | | | between the abdominal wall and the bottom of |
| | | | the cecum, making spot connections in |
| | | | between. The pocket turned out to be the |
| | | | wrinkled sheet surrounded by adipose tissue. |
| | 17 | 1 | Adipose tissue was formed between cecum and |
| | | | the abdomen wall making spot connections. |
| | | | The sheet had moved to the front surface of the |
| | | | cecum, and had partial overlap with the liver. |
| | 18 | 0 | Adipose tissue was formed between cecum and |
| | | | the abdomen wall making spot connections. |
| | | | The sheet had moved up to the left side of the |
| | | | abdomen, and had partial overlap with the |
| | | | liver. |
| | 19 | 3 | A light yellow coloured pocket was found |
| | | | between the abdominal wall and the bottom of |
| | | | the cecum, making tight connections in |
| | | | between. The pocket turned out to be the |
| | | | wrinkled sheet surrounded by adipose tissue. |
| | 20 | NA | Died post-surgery |
| | Mean | 0.89 | Average |
| | SD | 1.17 | Standard Deviation |
| | SEM | 0.39 | Standard Error of the Mean |
| Statistical | TTEST | 1.84E−06 | 2 samples equal variance T-test |
| Analysis | TTEST | 4.37E−05 | 2 samples unequal variance T-test |
| | Significance | p < 0.0001 | Reject null hypothesis --> Significance |

Results: The results indicate that adhesion barrier film effectively inhibits adhesion formation between traumatized cecum and adjacent abdominal wall at the site of injury. Five of the nine rats (rat no. 6, 8, 9, 10 and 18) treated with adhesion barrier film displayed complete inhibition of adhesion formation at the site of injury in contrast to formation of thick planar adhesion at the injury sites for the controls. Adhesion formation was partially inhibited in the one remaining adhesion barrier film-treated animal.

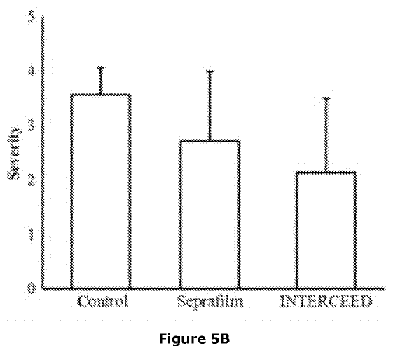

Comparison studies: Adhesion scores at six weeks after surgery are shown in FIGS. 5A and 5B. With regard to the extent of adhesion, the scores (means±SD, n=7 in each group) were 3.6±1.0 in the non-treated, 2.9±1.4 in the Seprafilm and 2.7±1.3 in the INTERCEED group. There were no significant differences in the extent of adhesion between the three treatment groups. The Seprafilm and INTERCEED groups showed no significant anti-adhesive effects compared to the control group.

Regarding the severity of adhesion, the scores were 3.6±0.5 in the control, 2.7±1.3 in the Seprafilm, and 2.1±1.4 in the INTERCEED group. There were no significant differences in adhesion severity between these groups. The Seprafilm and INTERCEED groups showed no significant anti-adhesive effects compared to the control group.

In the Seprafilm and INTERCEED groups, the local inflammation was so substantial that regeneration of the peritoneum was incomplete even at three weeks after surgery, indicating that the progress of adhesion continued until peritoneum regeneration was complete.

Local bleeding, insufficient haemostasis, or re-bleeding after surgery attenuates the anti-adhesive effects of INTERCEED. Seprafilm also has no haemostatic effect.

It will be appreciated that many further modifications and permutations of various aspects of the described embodiments are possible. Accordingly, the described aspects are intended to embrace all such alterations, modifications, and variations that fall within the spirit and scope of the appended claims.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

1.-34. (canceled)

35. A method of reducing the formation of post-surgery tissue adhesion and/or preventing the formation of tissue adhesion during or post-surgery in a subject in need thereof by administering an adhesion barrier composition, the adhesion barrier composition comprising:
a) sporopollenin; and
b) a biodegradable polymer; wherein
the weight ratio of sporopollenin is about 1 wt/wt % to about 80 wt/wt % relative to the total composition.

36. The method of claim 35, wherein the surgery is selected from peritoneal, pericardial, obstetric, gynecological, neurosurgical, arthroscopic, orthopedic, plastic, reconstructive, muscle or tendon surgery.

37. An adhesion barrier composition comprising:
a) sporopollenin; and
b) a biodegradable polymer; wherein
the weight ratio of sporopollenin is about 1 wt/wt % to about 80 wt/wt % relative to the total composition, or preferably about 20 wt/wt % to about 80 wt/wt % relative to the total composition.

38. The adhesion barrier composition of claim 37, wherein the sporopollenin comprises at least one of the following:
a) a polymer having ester bonds, wherein the sporopollenin is characterized with having at least 2% of the ester bond hydrolyzed;
b) a polymer having ether bonds, wherein the sporopollenin is characterized with having at least 2% of the ether bond hydrolyzed;
c) a polymer having peptide bonds, wherein at least 2% of the peptide bond is hydrolyzed; and
d) a lipid, wherein at least 2% of the lipid is saponificated.

39. The adhesion barrier composition of claim 37, wherein the sporopollenin is derived from a pollen, the pollen originating from a flowering plant of the genus Baccharis, Helianthus or Camellia, Coreopsis, Callistephus, Dahalia, Solidago or a combination thereof.

40. The adhesion barrier composition of claim 37, wherein the sporopollenin has a particle size of about 20 μm to about 50 μm.

41. The adhesion barrier composition of claim 37, wherein the biodegradable polymer is selected from poly(glycolic) acid, polylactic acid, polydioxanone, polycaprolactone, calcium alginate, sodium hyalurate, hyaluronic acid or a combination thereof, and preferably polylactic acid (PLA).

42. The adhesion barrier composition of claim 37, further comprising an aqueous medium.

43. The adhesion barrier composition of claim 37, further comprising a lipid selected from lauric acid, palmitic acid, α-linolenic acid, myristic acid, stearic acid, oleic acid, linoleic acid, eicosenoic acid, glycerides or a combination thereof.

44. The adhesion barrier composition of claim 37, further comprising pollenkitt.

45. The adhesion barrier composition of claim 44, wherein the pollenkitt is selected from an angiosperm.

46. The adhesion barrier composition of claim 37, further comprising intine selected from polysaccharides, cellulose, hemicellulose and pectin, or a combination thereof.

47. The adhesion barrier composition of claim 37, further comprising phenolic compound selected from gallic acid, caffeic acid, ferulic acid, p-coumaric, benzoic acid or a combination thereof.

48. The adhesion barrier composition of claim 47, wherein a weight ratio of the phenolic compound is at least 1 wt/wt % relative to the total composition.

49. The adhesion barrier composition of claim 37, wherein the adhesion barrier composition is capable of being completely resorbed after 6 months on application.

50. The adhesion barrier composition of claim 37, wherein the adhesion barrier composition is a film, gel or a liquid.

51. The adhesion barrier composition of claim 37, wherein when the adhesion barrier composition is an adhesion barrier film, the adhesion barrier film comprising:
a) a coating of sporopollenin; and
b) a biodegradable polymer film having two surfaces; wherein
the weight ratio of sporopollenin is about 1 wt/wt % to about 80 wt/wt % relative to the total composition; and wherein
sporopollenin coats at least one surface of the biodegradable polymer film.

52. The adhesion barrier composition of claim 51, wherein the surface of the biodegradable polymer film is 100% covered by sporopollenin.

53. The adhesion barrier composition of claim 51, wherein the coating of sporopollenin has a thickness of about 50 μm to about 200 μm.

54. A method of fabricating an adhesion barrier composition, comprising:
a) dispersing a sporopollenin in or on a biodegradable polymer; wherein
the weight ratio of sporopollenin is about 10 wt/wt % to about 80 wt/wt % relative to the total composition.
